(19) United States
(12) Patent Application Publication
(10) Pub. No.: US 2013/0141634 A1
(43) Pub. Date: Jun. 6, 2013

(54) IMAGING DEVICE

(71) Applicants: Tsuguhiro Korenaga, Osaka (JP); Norihiro Imamura, Osaka (JP)

(72) Inventors: Tsuguhiro Korenaga, Osaka (JP); Norihiro Imamura, Osaka (JP)

(21) Appl. No.: 13/701,924

(22) PCT Filed: May 18, 2012
(86) PCT No.: PCT/JP2012/003286

§ 371 Date: Dec. 4, 2012

(30) Foreign Application Priority Data
Jun. 27, 2011 (JP) 2011-142370

(51) Int. Cl. H04N 5/238

(52) U.S. Cl. CPC H04N 5/238
USPC 348/360

(57) ABSTRACT

An imaging device includes: a lens optical system including a first region and a second region having different optical properties; an imaging element including first pixels and second pixels; an arrayed optical element which is provided between the lens optical system and the imaging element, allows light passing through the first region to enter the first pixels, and allows light passing through the second region to enter the second pixels; a signal processing unit configured to generate object information using pixel values obtained from the first pixels and the second pixels; and a diffractive optical element provided between the arrayed optical element and the lens optical system and including a diffraction grating symmetrical about an optical axis of the lens optical system.

### TECHNICAL FIELD

The present invention relates to an imaging device such as a camera.

### BACKGROUND ART

There is an increasing need for imaging devices having not only the function of capturing two-dimensional images but also other functions. For example, there is a growing need for cameras having another function such as: measuring a distance to an object; capturing images at different wavelength bands such as an image at a visible wavelength and an image at an infrared wavelength; imaging a close object and a far object with high sharpness (increasing the depth of field); or capturing an image having a wide dynamic range.

An example of a method for the above-mentioned function of measuring a distance to an object is a method for which parallax information detected from images captured using more than one imaging optical system is used. The Depth From Defocus (DFD) method is known as a method for measuring a distance from a single imaging optical system to an object. The DFD method is a technique of calculating the distance based on an analysis of an amount of blur in captured images. In this method, the distance is estimated using more than one image because with a single image, it is impossible to determine whether the blur is the pattern of the object or is caused by the object distance (see Patent Literature (PTL) 1 and Non-Patent Literature (NPL) 1).

Furthermore, as a method for capturing images at different wavelength bands, a technique is disclosed which is to capture images by sequentially turning on white light and predetermined narrowband light, for example.

Moreover, as a method for capturing an image having a wide dynamic range, PTL 3 discloses a method by which a logarithmic conversion imaging device corrects non-uniform sensitivities of pixels by subtracting, from data obtained from each pixel, data which had been obtained by uniform light illumination and stored in a memory. PTL 4 discloses a method in which an optical path is separated using a prism and imaging is performed while varying the capturing condition (amount of light exposure) using two imaging elements. In the method of capturing images with different exposure time by time division and combining the captured images, images of an object are captured by time division, thus causing a problem of image discontinuities because when the object is moving, the images are not continuous due to the time difference. PTL 5 discloses a technique of correcting image discontinuities that occur in such a method.

### CITATION LIST

### Patent Literature

[PTL 1] Japanese Patent No. 3110095

[PTL 2] Japanese Patent No. 4253550

[PTL 3] Japanese Unexamined Patent Application Publication No. 05-30350

[PTL 4] Japanese Unexamined Patent Application Publication No. 2009-31682

[PTL 5] Japanese Unexamined Patent Application Publication No. 2002-101347

### Non Patent Literature

[NPL 1] Xue Tu, Youn-sik Kang and Murali Subbarao Two- and Three-Dimensional Methods for Inspection and Metrology V. Edited by Huang, Peisen S. Proceedings of the SPIE, Volume 6762, pp. 676203 (2007).

### SUMMARY OF INVENTION

### Technical Problem

An object is to provide an imaging device which can achieve not only the function of capturing two-dimensional images but also at least one of other functions including the above-described functions (e.g., measuring an object distance, capturing images at different wavelength bands, increasing the depth of field, and capturing an image having a wide dynamic range).

### Solution to Problem

An imaging device according to an aspect of the present invention includes: a lens optical system including at least a first region and a second region having different optical properties; an imaging element including at least first pixels and second pixels which light passing through the lens optical system enters; an arrayed optical element which is provided between the lens optical system and the imaging element, allows light passing through the first region to enter the first pixels, and allows light passing through the second region to enter the second pixels; a signal processing unit configured to generate object information using first pixel values obtained from the first pixels and second pixel values obtained from the second pixels; and a diffractive optical element provided between the arrayed optical element and the lens optical system and including a diffraction grating symmetrical about an optical axis of the lens optical system.

### Advantageous Effects of Invention

According to the present invention, it is possible to achieve not only the function of capturing two-dimensional images but also at least one of other functions (e.g., measuring an object distance, capturing images at different wavelength bands, increasing the depth of field, and capturing an image having a wide dynamic range). The present invention requires neither a special imaging element nor a plurality of imaging elements.

### BRIEF DESCRIPTION OF DRAWINGS

FIG. 1 is a schematic diagram showing a configuration of an imaging device according to Embodiment 1 of the present invention.

FIG. 2 is a front view of a first optical element according to Embodiment 1 of the present invention, seen from the object side.

FIG. 3 is a configuration diagram of a third optical element according to Embodiment 1 of the present invention.

FIG. 4 is a diagram for describing a positional relationship between a third optical element and pixels on an imaging element according to Embodiment 1of the present invention.

FIG. 5 is a diagram showing spherical aberrations of light fluxes passing through a first region and a second region according to Embodiment 1 of the present invention.

FIG. 6 is a graph showing a relationship between object distance and sharpness according to Embodiment 1 of the present invention.

FIG. 7 is a diagram showing light rays collected at a position away from the optical axis by a distance H according to Embodiment 1 of the present invention.

FIG. 8 is a diagram for describing a path of a chief ray according to Embodiment 1 of the present invention.

FIG. 9 is a diagram showing analyses of paths of light fluxes including a chief ray entering a lenticular lens at an incident angle according to Embodiment 1 of the present invention.

FIG. 10 is a diagram showing an image-side telecentric optical system.

FIG. 11 is a diagram for describing a positional relationship between a third optical element and an imaging element according to Embodiment 2 of the present invention.

FIG. 12 is a diagram showing analyses of paths of light fluxes including a chief ray entering a lenticular lens at an incident angle θ according to Embodiment 2 of the present invention.

FIG. 13 is a front view of a first optical element according to Embodiment 3 of the present invention, seen from the object side.

FIG. 14 is a configuration diagram of a third optical element according to Embodiment 3 of the present invention.

FIG. 15 is a diagram for describing a positional relationship between a third optical element and pixels on an imaging element according to Embodiment 3 of the present invention.

FIG. 16 is a graph showing a relationship between object distance and sharpness according to Embodiment 3 of the present invention.

FIG. 17 is a diagram for describing a third optical element according to Embodiment 4 of the present invention.

FIG. 18 is a diagram for describing wavelength dependency of the first-order diffraction efficiency of a blazed diffraction grating according to Embodiment 4 of the present invention.

FIG. 19 is an enlarged cross-section diagram of a third optical element and an imaging element according to Embodiment 5 of the present invention.

FIG. 20 is an enlarged cross-section diagram of a third optical element and an imaging element according to a variation of Embodiment 5 of the present invention.

FIG. 21 is a cross-section diagram of a third optical element according to a variation of the present invention.

### DESCRIPTION OF EMBODIMENTS

When a distance to an object is to be obtained using the above-described conventional techniques, use of more than one imaging optical system results in an increase in size and cost of the imaging device. Furthermore, the manufacture is made difficult due to the need to make the properties of the imaging optical systems uniform with each other and to make the optical axes of two imaging optical systems in parallel with high precision. In addition, a large number of man-hours are required due to the need of calibration for determining camera parameters.

The DFD method disclosed in PTL 1 and NPL 1 allows calculation of a distance to an object using a single imaging optical system. However, the method of PTL 1 and NPL 1 requires capturing a plurality of images by time division while varying the distance to the object at which the object is in focus (focus distance). Applying such a technique to moving pictures causes image discontinuities due to differences in the capturing time, thereby resulting in a problem of lower precision in the distance measuring.

PTL 1 discloses an imaging device which separates an optical path using a prism and capturing an image using two image planes having different back focuses so that a distance to an object can be measured in one imaging operation. However, such a technique requires two image planes, resulting in a problem of an increased size of the imaging device and a significant increase in cost.

When images at different wavelength bands are to be captured using the technique disclosed in PTL 2, a white light source and a predetermined narrowband light source are sequentially turned on to capture images by time division. With this technique, capturing images of a moving object causes color inconsistencies due to the time differences.

When an image having a wide dynamic range is to be captured, a method of performing logarithmic conversion on received signals requires a circuit for performing the logarithmic conversion on a pixel signal on a pixel-by-pixel basis, which hinders reduction of the pixel size. Furthermore, the technique disclosed in PTL 1 requires a means for recording correction data used for correcting non-uniform sensitivities of the pixels, thereby increasing the cost.

Moreover, the technique of PTL 2 requires two imaging elements, resulting in an increase in size of the imaging device and a significant increase in cost.

PTL 3 discloses a technique of correcting image discontinuities, but it is theoretically difficult to completely correct the image discontinuities caused by time differences for various moving objects.

The present invention can achieve, in one imaging operation using a single imaging optical system, not only the function of capturing two-dimensional images but also at least one of other functions (e.g., measuring an object distance, capturing images at different wavelength bands, increasing the depth of field, and capturing an image having a wide dynamic range). The present invention requires neither a special imaging element nor a plurality of imaging elements.

The following describes an imaging device according to embodiments of the present invention while referring to the drawings.

It is to be noted that any of the following embodiments is to show a specific example of the present invention. The numerical values, shapes, materials, structural elements, the arrangement and connection of the structural elements, steps, the processing order of the steps etc. shown in the following embodiments are mere examples, and are thus not intended to limit the present invention. Furthermore, among the structural elements described in the following embodiments, those not recited in the independent claims indicating the most generic concept are described as arbitrary structural elements.

### Embodiment 1

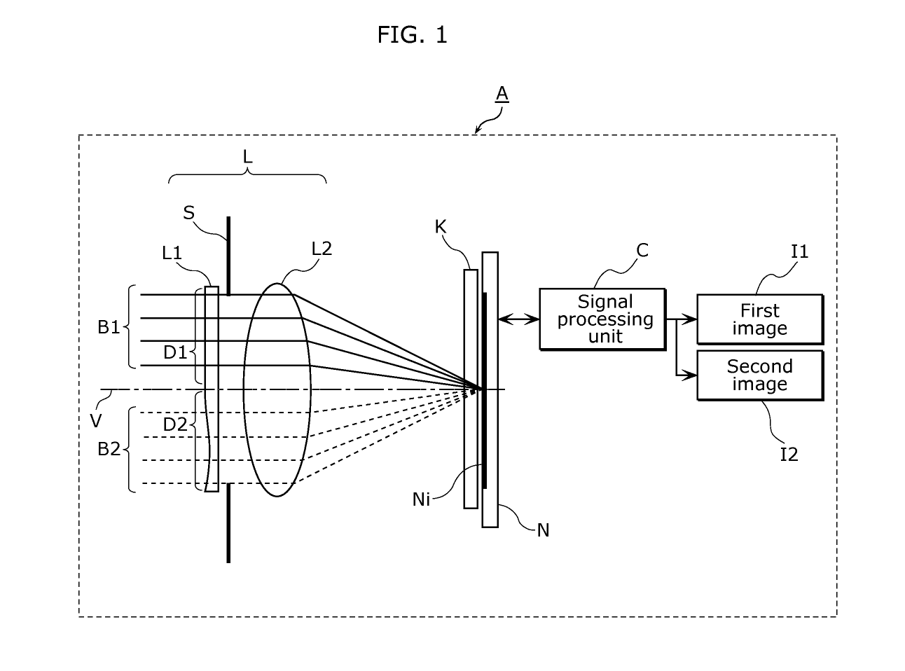

FIG. 1 is a schematic diagram showing a configuration of an imaging device A according to Embodiment 1. The imaging device A according to the present embodiment includes a lens optical system L, a third optical element K provided near a focal point of the lens optical system L, an imaging element N, and a signal processing unit C.

The lens optical system L includes a first region D1 and a second region D2 each of which a light flux B1 or B2 from an object (not shown) enters, and which have different optical properties. Here, the optical properties refer to, for example, focus properties, the wavelength band of transmitted light, or light transmittance, or a combination of these.

Furthermore, to have different focus properties is to have a difference in at least one of the properties contributing to collection of light in the optical system, and is specifically to have a difference in, for example, a focal length, a distance to an object at which the object is in focus, and a range of distance at which the sharpness is at a predetermined level or higher. Adjusting at least one of the curvature radius, spherical aberration properties, and refractive index allows the first region D1 and the second region D2 to have different focus properties.

The lens optical system L includes a first optical element L1, a diaphragm S having an opening in a region including an optical axis V of the lens optical system L, and a second optical element L2.

The first optical element L1 is provided near the diaphragm S and includes the first region D1 and the second region D2 having different optical properties.

In FIG. 1, the light flux B1 passes through the first region D1 of the first optical element L1, whereas the light flux B2 passes through the second region D2 of the first optical element L1. The light fluxes B1 and B2 pass through the first optical element L1, the diaphragm S, the second optical element L2, and the third optical element K in this order, and reach an image plane Ni of the imaging element N.

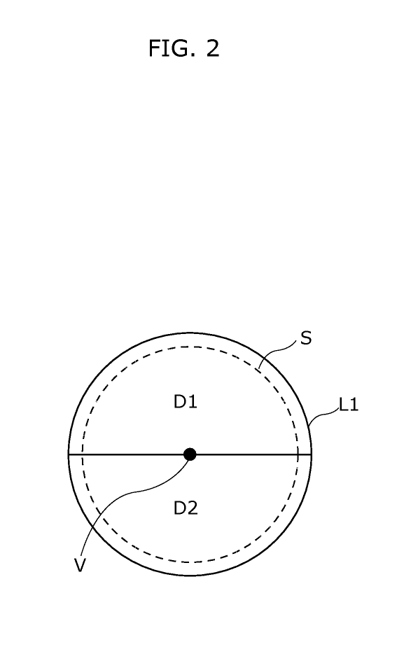

FIG. 2 is a front view of the first optical element L1 seen from the object side. The first region D1 and the second region D2 are obtained by dividing a plane vertical to the optical axis V into two upper and lower regions with the optical axis V as the center of the boundary.

The second optical element L2 is a lens that the light transmitted by the first optical element L1 enters. In FIG. 1, the second optical element L2 includes one lens, but it may include a plurality of lenses. Furthermore, the second optical element L2 may be integrally formed with the first optical element L1. Such a case simplifies the alignment of the first optical element L1 and the second optical element L2 at the time of manufacture.

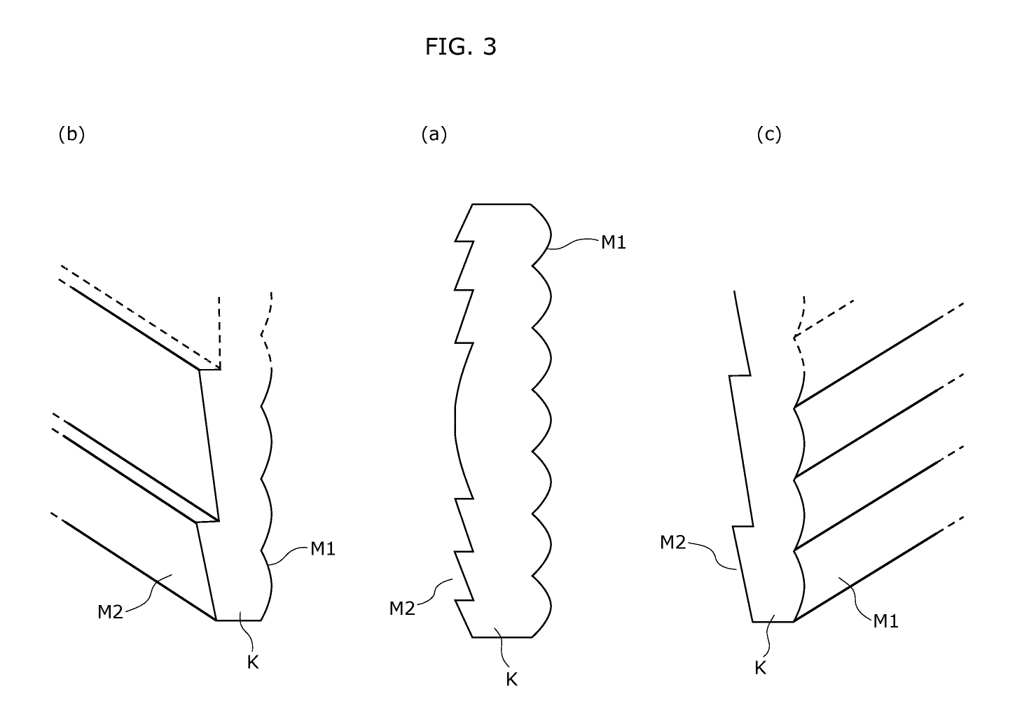

FIG. 3 is a configuration diagram of the third optical element K. More specifically, the part (a) of FIG. 3 is a cross-section diagram of the third optical element K. The part (b) of FIG. 3 is a partial enlarged perspective view of the third optical element K seen from a blazed diffraction grating M2 side. The part (c) of FIG. 3 is a partial enlarged perspective view of the third optical element K seen from a lenticular lens M1 side. It is to be noted that the shape and the exact size of the pitch of each of the lenticular lens M1 and the blazed diffraction grating M2 will not be described because it is sufficient as long as they are determined according to the function or use purpose of the imaging device N.

On a surface of the third optical element K on the imaging element N side, a lenticular lens M1 is formed which includes elongate optical elements (convex lenses) having an arc cross section protruding on the imaging element N side and arranged in the vertical direction (column direction). The lenticular lens M1 is equivalent to an arrayed optical element.

Furthermore, on a surface of the third optical element K on the lens optical system L side (i.e., on the object side), a blazed diffraction grating M2 is formed which is symmetric about the optical axis V. That is to say, the third optical element K is an optical element into which a diffractive optical element having a diffraction grating symmetric about the optical axis V and the arrayed optical element are integrated. In other words, in the present embodiment, the diffractive optical element and the arrayed optical element are integrally formed. As described, integrally forming the arrayed optical element and the diffractive optical element makes easier the alignment of the arrayed optical element and the diffractive optical element at the time of manufacture. It is to be noted that the arrayed optical element and the diffractive optical element do not necessarily have to be formed integrally, and may be provided as separate optical elements.

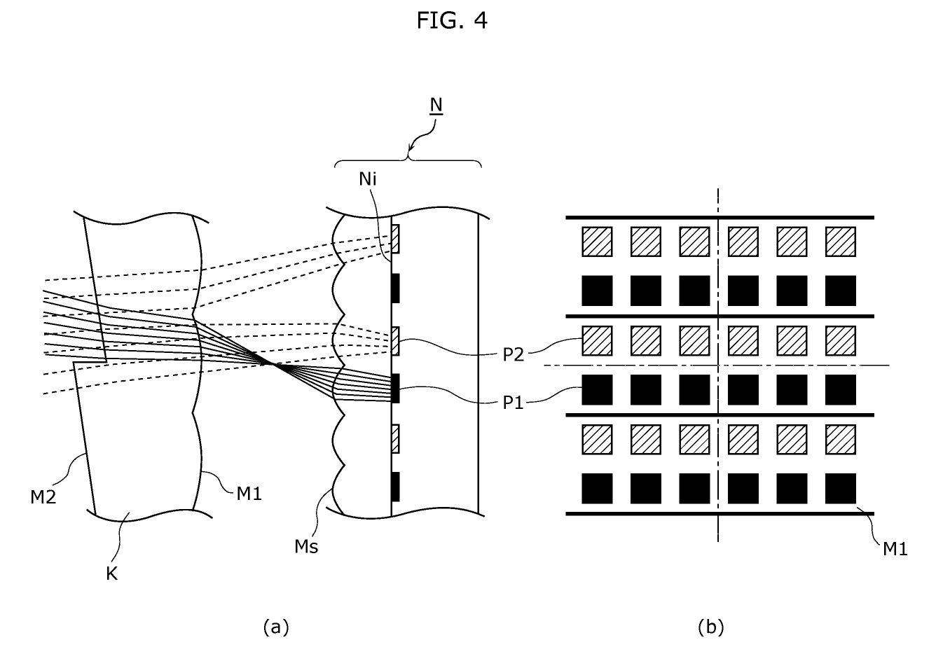

FIG. 4 is a diagram for describing the positional relationship between the third optical element K and pixels on the imaging element N. More specifically, the part (a) of FIG. 4 is an enlarged view of the third optical element K and the imaging element N. Furthermore, the part (b) of FIG. 4 is a diagram showing the positional relationship between the third optical element K and the pixels on the imaging element N.

The third optical element K is provided near a focal point of the lens optical system L and is provided at a position away from the image plane Ni at a predetermined distance. Furthermore, pixels are arranged in rows and columns on the image plane Ni of the imaging element N. Each of these pixels arranged in such a manner can be classified as a first pixel P1 or a second pixel P2.

In the present embodiment, the first pixels P1 are arranged in a row in the horizontal direction (row direction) and the second pixels P2 are arranged in a row in the horizontal direction. In the vertical direction (column direction), the first pixels P1 and the second pixels P2 are alternately arranged. Furthermore, a microlens Ms is provided over the first pixels P1 and the second pixels P2.

Moreover, each of the optical components included in the lenticular lens M1 has a one-to-one correspondence with a pair of a row of the first pixels P1 and a row of the second pixels P2 on the image plane Ni.

With such a structure, large part of the light flux B1 (solid lines in FIG. 1) passing through the first region D1 on the first optical element L1 shown in FIG. 2 reaches the first pixels P1 on the image plane Ni, while large part of the light flux B2 (dashed lines in FIG. 1) passing through the second region D2 reaches the second pixels P2 on the image plane Ni.

More specifically, the third optical element K allows the light flux B1 passing through the first region D1 to enter the first pixels P1 and allows the light flux B2 passing through the second region D2 to enter the second pixels P2 when parameters such as the following are appropriately set: the refractive index of the third optical element K, the distance from the image plane Ni to the third optical element K, the diffraction pitch of the blazed diffraction grating M2, and the curvature radius of the surface of the lenticular lens M1.

With a typical imaging optical system, the angle of a light ray at a focal point is determined based on the position at which the light ray has passed through the diaphragm. Thus, by providing, near the diaphragm, the first optical element P1 including the first region D1 and the second region D2, and providing the third optical element K near the focal point as described above, it is possible of separately guide the light flux B1 and the light flux B2 passing through the corresponding regions, to the first pixels P1 and the second pixels P2, respectively.

Here, the signal processing unit C shown in FIG. 1 generates object information using first pixel values obtained from the first pixels P1 and second pixel values obtained from the second pixels P2. In the present embodiment, the signal processing unit C generates, as the object information, a first image I1 including the first pixel values and a second image I2 including the second pixel values.

The first image I1 and the second image I2 are images respectively obtained from the light flux B1 and the light flux B2 passing through the first region D1 and the second region D2 having different optical properties. For example, when the first region D1 and the second region D2 have such optical properties that make the focus properties of the passing light rays different from each other, luminance information of the first image I1 and luminance information of the second image I2 indicate different properties depending on a change in the object distance. By using this difference, a distance to an object can be determined. That is to say, a distance to an object can be obtained in one imaging operation using a single imaging system. The details will be described later.

Furthermore, the depth of field can be increased by generating an output image using a sharper one of the first image I1 and the second image I2 that are obtained using the first region D1 and the second region D2 having different focus properties.

Moreover, when the first region D1 and the second region D2 are different in wavelength band of passing light, the first image I1 and the second image I2 are images obtained from light at different wavelength bands. For example, the first region D1 is assumed to be an optical filter having properties of transmitting visible light and substantially blocking near-infrared light. The second region D2 is assumed to be an optical filter having properties of substantially blocking visible light and transmitting near-infrared light. This enables implementation of a day- and night-vision imaging device and an imaging device for biometric authentication. That is to say, an arbitrary multispectral image can be captured in one imaging operation using a single imaging system.

Furthermore, when the first region D1 and the second region D2 are different in transmittance, the amount of exposure of the first pixels P1 and the amount of exposure of the second pixels P2 are different. For example, suppose a case where the transmittance of the second region D2 is higher than the transmittance of the first region D1. Even when an amount of light greater than can be detected is supplied to the first pixels P1 (i.e., when the pixel values of the first pixels P1 are saturated), an accurate object brightness can be calculated using values detected from the second pixels P2. In contrast, when an amount of light within the largest amount of light detectable by the first pixels P1 is supplied to the first pixels P1 (i.e., when the pixel values of the first pixels P1 are not saturated), values detected from the first pixels P1 can be used. That is to say, an image having a wide dynamic range can be captured in one imaging operation using a single imaging system.

In such a manner, the imaging device A generates different images by allowing light passing through the first region D1 and the second region D2 having different optical properties, to enter different pixels. The difference in the optical properties between the first region D1 and the second region D2 results in a difference in the object information between the generated images. By using the difference in the object information, it is possible to achieve functions such as measuring an object distance, capturing images at different wavelength bands, increasing the depth of field, and capturing an image having a wide dynamic range. That is to say, the imaging device A can achieve not only the function of merely capturing two-dimensional images but also other functions in one imaging operation using a single imaging system.

It is to be noted that the optical properties different between the first region D1 and the second region D2 are not limited to the above-described example.

Next, a method for determining an object distance from the object information will be described in detail as an example of use of the object information.

Here, the surface of the first optical element L1 on the object side includes the first region D1 which is a flat surface and the second region D2 which is an optical surface having a point spread function approximately constant along the optical axis direction in a predetermined region near the focal point of the lens optical system L. Furthermore, the f-number of the second lens L2 is 2.8.

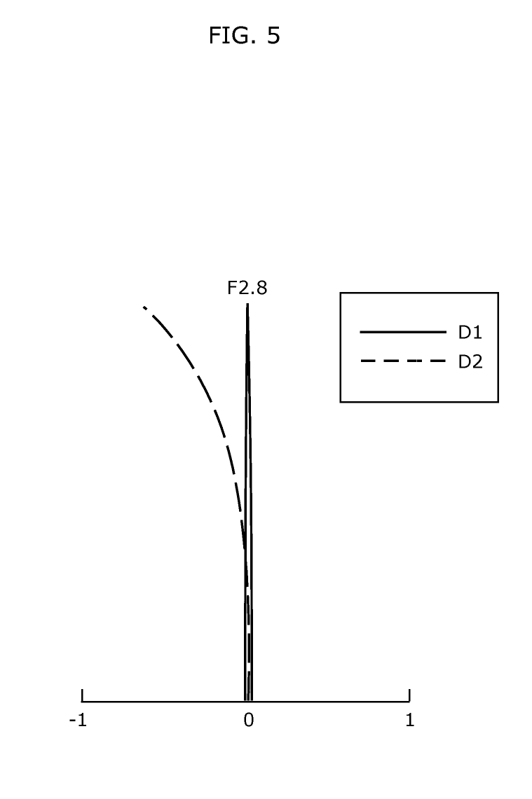

FIG. 5 is a diagram showing spherical aberrations of the light fluxes passing through the first region D1 and the second region D2 according to the present embodiment. Here, the first region D1 is designed so as to reduce the spherical aberration of the light flux passing through the first region D1. On the other hand, the second region D2 is intentionally designed so as to increase the spherical aberration of the light flux passing through the second region D2.

Adjusting the properties of the spherical aberration caused by the second region D2 allows the point spread function of the image generated by the light flux passing through the second region D2 to be approximately constant in the predetermined region near the focal point of the lens optical system L. That is to say, the point spread function of the image can be made approximately constant even when the object distance changes.

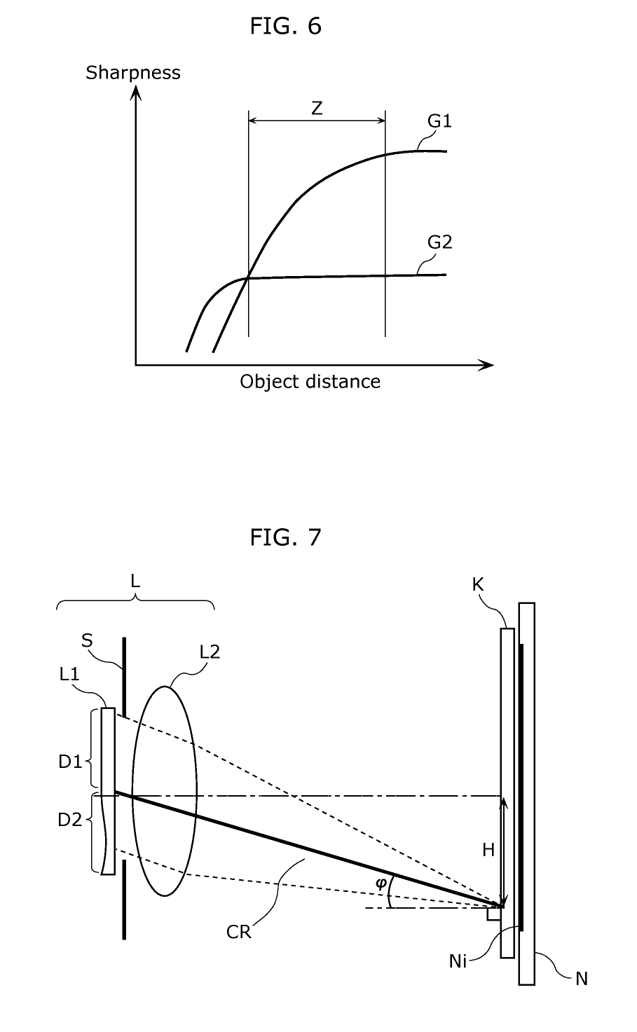

Because the image sharpness increases as the size of the point image in the point spread function decreases, the relationship between the object distance and the sharpness is as shown in FIG. 6.

FIG. 6 is a graph showing the relationship between the object distance and the sharpness according to the present embodiment. In the graph shown in FIG. 6, a profile G1 shows the sharpness of a predetermined region of the image generated using the pixel values of the first pixels P1, while a profile G2 shows the sharpness of a predetermined region of the image generated using the pixel values of the second pixels P2. The sharpness can be determined using a difference between luminance values of pixels adjacent to each other in an image block of a predetermined size. Furthermore, the sharpness can also be determined based on a frequency spectrum obtained through Fourier transform on the luminance distribution of an image block of a predetermined size.

A range Z is a range in which the sharpness according to the profile G1 varies in response to a change in the object distance and is a range in which the sharpness according to the profile G2 hardly varies even when the object distance changes. Thus, the object distance can be determined using such a relationship in the range Z.

For example, in the range Z, a ratio between the sharpness according to the profile G1 and the sharpness according to the profile G2 is correlated with the object distance. In view of this, use of such a correlation enables determination of the object distance based on the ratio between the sharpness of the image generated using only the pixel values of the first pixels P1 and the sharpness of the image generated using only the pixel values of the second pixels P2.

It is to be noted that such determination of the object distance is an example of use of the object information, and an image such as an image having a wide dynamic range or an image having a large depth of field may be generated using the object information. Furthermore, the signal processing unit C may, using the object information, determine the object distance or generate an image such as an image having a wide dynamic range or an image having a large depth of field.

Next, the following describes an advantageous effect of the blazed diffraction grating M2 formed on the third optical element K on the lens optical system L side (i.e., on the object side) shown in FIG. 3.

FIG. 7 is a diagram showing light rays collected at a position away from the optical axis V by a distance H according to the present embodiment. In FIG. 7, an angle (an incident angle with respect to the surface of the third optical element K on the object side) between a chief ray CR (a light ray passing through the center of the diaphragm S) and the optical axis V is φ. When the distance H is to be varied as a parameter, there is one chief ray CR and one incident angle φ for each distance H. When the distance H is 0, the incident angle φ is 0. In a typical imaging lens optical system, the incident angle φ increases with the distance H.

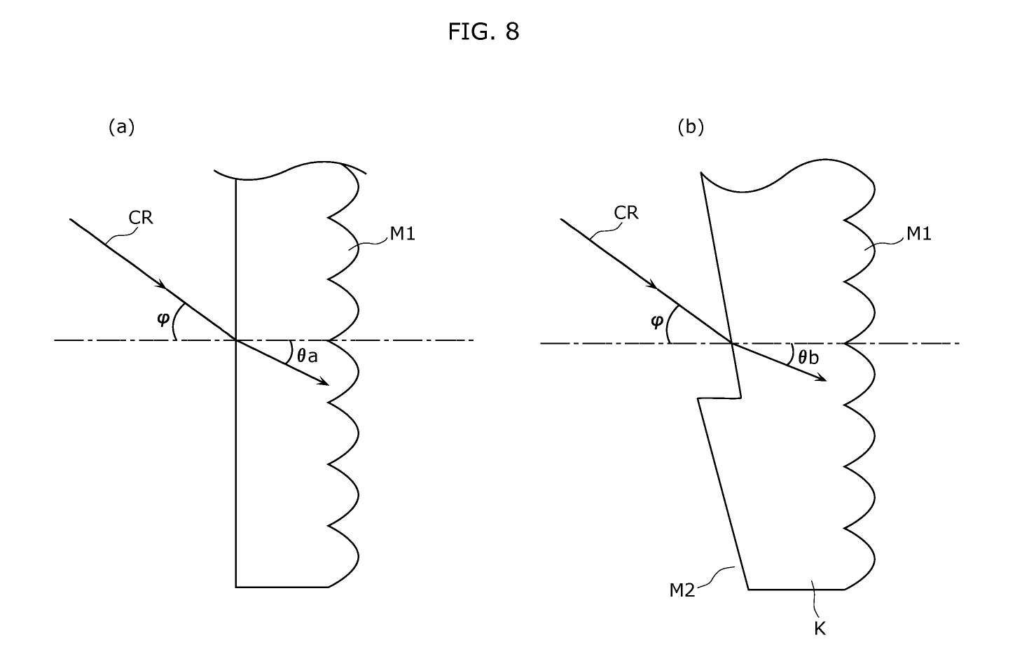

FIG. 8 is a diagram showing a path of the chief ray CR at a position away from the optical axis V by the distance H. More specifically, the part (a) of FIG. 8 shows a path of the chief ray CR in a comparable optical element in which the blazed diffraction grating M2 is not formed. The part (b) of FIG. 8 shows a path of the chief ray CR in the third optical element K in which the blazed diffraction grating M2 according to the present embodiment is formed.

In the part (a) of FIG. 8, the chief ray CR refracts on the incident plane of the comparable optical element having a refractive index of n, by an angle θa satisfying nsinθa=sinφ, before reaching the lenticular lens M1.

In contrast, in the part (b) of FIG. 8, the chief ray CR diffracts by an angle θb before reaching the lenticular lens M1. The angle θb is given by the equation below.

sin φ−n sin θb=mλ/P (Equation 1)

Here, λ denotes wavelength, m denotes diffraction order, and P denotes pitch of the blazed diffraction grating.

With the blazed diffraction grating M2, the condition under which the diffraction efficiency is theoretically 100% with respect to a light ray entering at an incident angle of 0° can be expressed by the equation below using the depth d of the diffraction grooves.

d=mλ/(n−1) (Equation 2)

In Equation 2, d equals 0.95 μm when λ=500 nm, m=1, and n=1.526.

The blazed diffraction grating M2 diffracts the incident light rays to change the wavefront. For example, with the condition under which Equation 2 is true, the blazed diffraction grating M2 allows the entire incident light to be m-th diffracted light, thereby changing the light direction.

The blazed diffraction grating M2 is one of phase gratings that achieve diffraction according to phase distribution determined by its shape. That is to say, the blazed diffraction grating M2 has a groove for every phase difference 2n corresponding to one wavelength based on the phase distribution for refracting light rays toward a desired direction. A Fresnel lens is an example of optical elements which are similar in shape to the blazed diffraction grating M2. The Fresnel lens is a planar lens fabricated by dividing a lens according to the distance from the optical axis and shifting the lens surface in the direction of the lens thickness. Thus, the Fresnel lens is different from the blazed diffraction grating M2 (phase grating). The Fresnel lens utilizes light refraction and thus its groove pitch is as large as several hundred micrometers to several millimeters. Furthermore, the Fresnel lens does not achieve large refraction of light rays which can be brought about by high-order diffraction where m is 2 or greater.

In the present embodiment, the incident light rays are refracted toward the optical axis when the blazed diffraction grating M2 has diffraction grooves formed toward the optical axis and has curved surfaces formed between the diffraction grooves toward the outer circumference as shown in the part (a) of FIG. 3. That is to say, in this case, the blazed diffraction grating M2 has a positive light-collecting power. This is equivalent to m being positive in Equation 1.

As in the present embodiment, θa>θb is true as a result of the formation of the blazed diffraction grating M2 having positive m on the surface of the third optical element K on the object side. This means that the third optical element K allows the direction of the light rays entering the lenticular lens M1 to be closer to the direction of the optical axis V, as compared to the comparable optical element in which the blazed diffraction grating M2 is not formed. As in the present embodiment, the blazed diffraction grating M2 allows the light to reach the lenticular lens M1 with an angle at which the light is closer to being parallel to the optical axis.

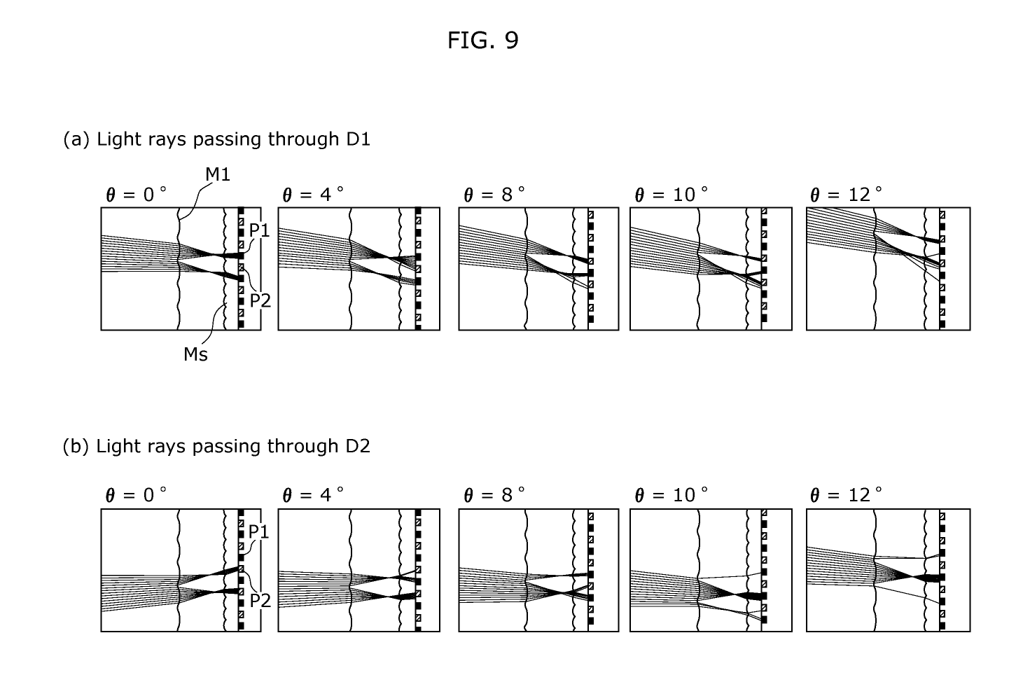

FIG. 9 is a diagram showing analyses of paths of light fluxes including the chief ray CR entering the lenticular lens M1 at an incident angle θ. FIG. 9 shows only the representative light rays including the chief ray CR.

The part (a) of FIG. 9 shows an analysis of the paths of light rays passing through the first region D1 of the first optical element L1. The part (b) of FIG. 9 shows an analysis of the paths of light rays passing through the second region D2 of the first optical element L1. The parts (a) and (b) of FIG. 9 show analyses when θ is 0°, 4°, 8°, 10°, or 12°.

As shown in the part (a) of FIG. 9, when θ=0°, the light rays passing through the first region D1 reach only the first pixels P1 and do not reach the second pixels P2. Furthermore, as shown in the part (b) of FIG. 9, when θ=0°, the light rays passing through the second region D2 reach only the second pixels P2 and do not reach the first pixels P1. This shows that when θ=0°, the light rays are properly separated by the lenticular lens M1, and no crosstalk occurs.

On the other hand, when θ≧4°, the light rays passing through the first region D1 reach the second pixels P2 as well as the first pixels P1, and the light rays passing through the second region D2 reach the first pixels P1 as well as the second pixels P2. This shows that when θ≧4°, the light rays are not properly separated by the lenticular lens M1, and crosstalk occurs. When the crosstalk occurs as in this case, there is significant deterioration of the quality of the image generated using the pixel values of the first pixels P1 and the image generated using the pixel values of the second pixels P2. This results in deterioration of the accuracy of various information (e.g., stereo information) generated using such images.

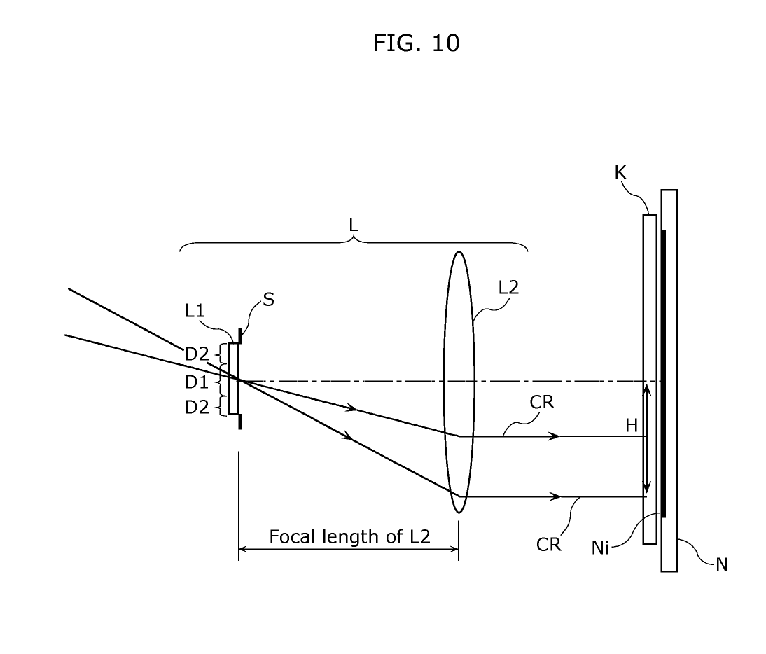

When the blazed diffraction grating M2 is not formed as in the comparable optical element shown in the part (a) of FIG. 8, θa<4° cannot be satisfied unless φ<6° is met according to the Snell's law of refraction. To satisfy φ<6° regardless of the distance H from the optical axis V, the lens optical system L needs to be an image-side telecentric optical system or a similar optical system as shown in FIG. 10.

The image-side telecentric optical system is an optical system in which the chief ray CR (arbitrary chief ray) is approximately parallel to the optical axis V regardless of the distance H as shown in FIG. 10. That is, it is an optical system in which the incident angle φ of the chief ray CR entering the surface of the third optical element K on the object side is approximately zero. The lens optical system L becomes the image-side telecentric optical system when the diaphragm S is provided at a position away from the principle point of the lens optical system L by a focal length f on the object side. Implementing the image-side telecentric optical system involves such a restriction on the position of the diaphragm S, thereby reducing the design flexibility of the imaging device. More specifically, implementing a telecentric optical system requires an increase in the size of the lens optical system or an increase in the number of lenses. A further increase in the size of the lens optical system or a further increase in the number of lenses is required particularly when the angle of view of the lens optical system needs to be increased.

In the present embodiment, the diffraction effect brought about by the blazed diffraction grating M2 formed on the surface of the third optical element K on the object side as shown in the part (b) of FIG. 8 allows reduction of the incident angle of the light rays to the lenticular lens M1 from the angle θa to the angle θb. In other words, the light rays entering the lenticular lens M1 can be made closer to being parallel to the optical axis.

As an example, it is assumed that the refractive index n of the third optical element K is 1.526 and the depth of the diffraction grooves of the blazed diffraction grating M2 is 0.95 μm. With this, Equation 2 gives that m is approximately 1 for light having a wavelength of 500 nm. This means that the blazed diffraction grating M2 allows generation of the first-order diffracted light with a diffraction efficiency of approximately 100%.

Given that the pitch of the diffraction grating at a position at which the chief ray CR enters the blazed diffraction grating M2 is 7 μm, θb is about 4° when φ is 10°. That is to say, the third optical element K having the blazed diffraction grating M2 can reduce the crosstalk as compared to the comparable optical element shown in the part (a) of FIG. 8, even when the incident angle φ of the chief ray CR entering the surface of the third optical element K on the object side increases by about 4°.

In other words, the imaging device A according to the present embodiment can reduce the crosstalk even when the incident angle φ of the chief ray CR to the lenticular lens M1 increases by degrees up to about 10°. Thus, the lens optical system L does not necessarily have to be an image-side telecentric optical system, and may be an image-side non-telecentric optical system.

As described thus far, the imaging device A according to the present embodiment can, using the lenticular lens M2, allow the light flux passing through the first region D1 to reach the first pixels P1, and allow the light flux passing through the second region D2 to reach the second pixels P2. Thus, the imaging device A can generate two images in one imaging operation using a single imaging optical system. Furthermore, providing the blazed diffraction grating M2 between the first optical element L1 and the lenticular lens M1 allows the direction of the light entering the lenticular lens M1 to be closer to the direction of the optical axis. This results in reduction of the crosstalk even when the lens optical system L is an image-side non-telecentric optical system, thereby increasing the design flexibility of the imaging device A. That is to say, the imaging device A according to the present embodiment can increase the design flexibility and reduce the crosstalk as well as being capable of generating a plurality of images in one imaging operation using a single imaging optical system.

Furthermore, with the imaging device A, the opening of the diaphragm S is formed in a region including the optical axis and the first optical element L1 is provided near the diaphragm, which is particularly desirable because such a structure allows a bright image to be captured with less light loss.

### Embodiment 2

Next, Embodiment 2 of the present invention will be described.

The lens optical system L can be further miniaturized and an imaging device smaller in size and larger in angle of view can be implemented if the crosstalk can be reduced even when the incident angle φ of the chief ray CR entering the surface of the third optical element K on the object side, that is, the angle between the chief ray CR and the optical axis V, is further increased.

In view of this, in the present embodiment, each optical component (convex lens) included in a lenticular lens M3 is offset in relation to the arrangement of corresponding first pixels P1 and second pixels P2. The following describes the imaging device A according to the present embodiment using a comparison with a comparable imaging device in which each optical component included in the lenticular lens M3 is not offset.

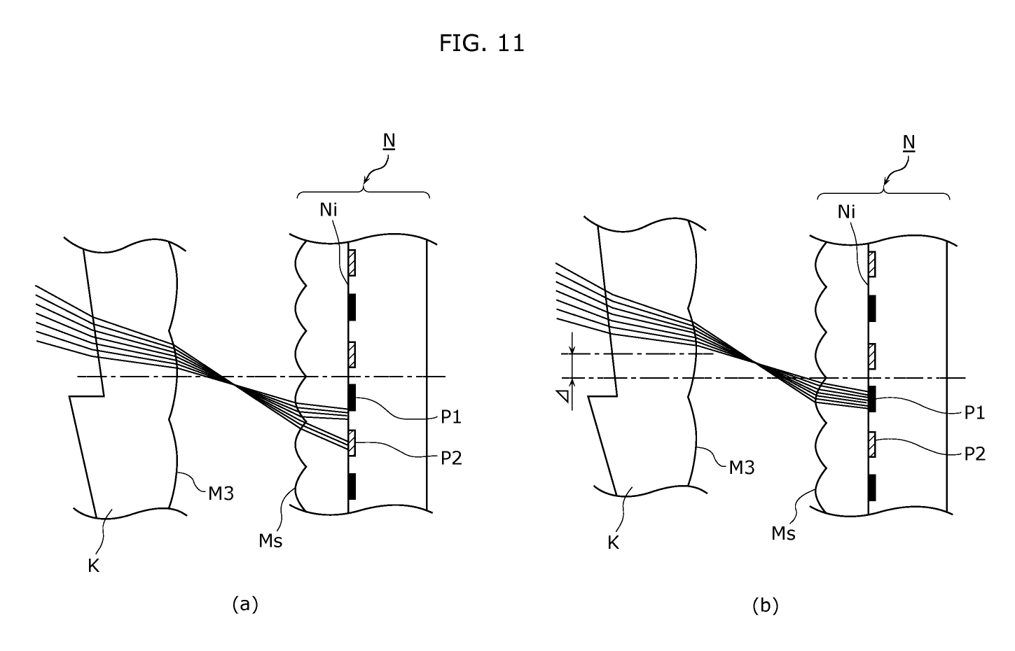

FIG. 11 is a diagram for describing the positional relationship between the third optical element K and the imaging element N according to the present embodiment. More specifically, the part (a) of FIG. 11 is an enlarged view of the third optical element K and the imaging element N at a position away from the optical axis of the comparable imaging device. The part (b) of FIG. 11 is an enlarged view of the third optical element K and the imaging element N at a position away from the optical axis of the imaging device according to Embodiment 2. The parts (a) and (b) of FIG. 11 show, among the light fluxes passing through the third optical element K, only the light flux passing through the first region D1.

As for the comparable imaging device shown in the part (a) of FIG. 11, each optical component included in the lenticular lens is not offset in relation to the arrangement of the corresponding first pixels P1 and second pixels P2. In other words, in the direction parallel to the optical axis, the center of each optical component matches the center of a pair of the corresponding first pixels P1 and second pixels P2.

With such a comparable imaging device, part of the light flux passing through the first region D1 reaches the second pixels P2 adjacent to the first pixels P1 as shown in the part (a) of FIG. 11. In other words, the crosstalk occurs at a position, away from the optical axis V, at which the incident angle φ of the light entering the third optical element K increases.

On the other hand, as for the imaging device A according to the present embodiment shown in the part (b) of FIG. 11, each optical component included in the lenticular lens M3 is offset in relation to the arrangement of the corresponding first pixels P1 and second pixels P2. In other words, in the direction parallel to the optical axis, the center of each optical component is displaced toward the optical axis V by an offset value Δ, from the center of the arrangement of a pair of the corresponding first pixels P1 and second pixels P2.

With such an imaging device A according to the present embodiment, the light flux passing through the first region D1 reaches only the first pixels P1 as shown in the part (b) of FIG. 11. That is to say, it is possible to reduce the crosstalk by offsetting, in relation to the pixel arrangement, each optical component included in the lenticular lens M3 of the third optical element K in a direction closer to the optical axis V by the offset value Δ as shown in the part (b) of FIG. 11.

It is to be noted that the incident angle φ of the light entering the surface of the third optical element K on the object side varies depending on the distance H from the optical axis V. Thus, it is sufficient as long as the offset value Δ is set according to the incident angle φ of the light flux entering the surface of the third optical element K on the object side. For example, it is sufficient as long as the lenticular lens M3 has the offset value Δ that increases with the distance from the optical axis V. This enables reduction of the crosstalk even when the distance from the optical axis V is large.

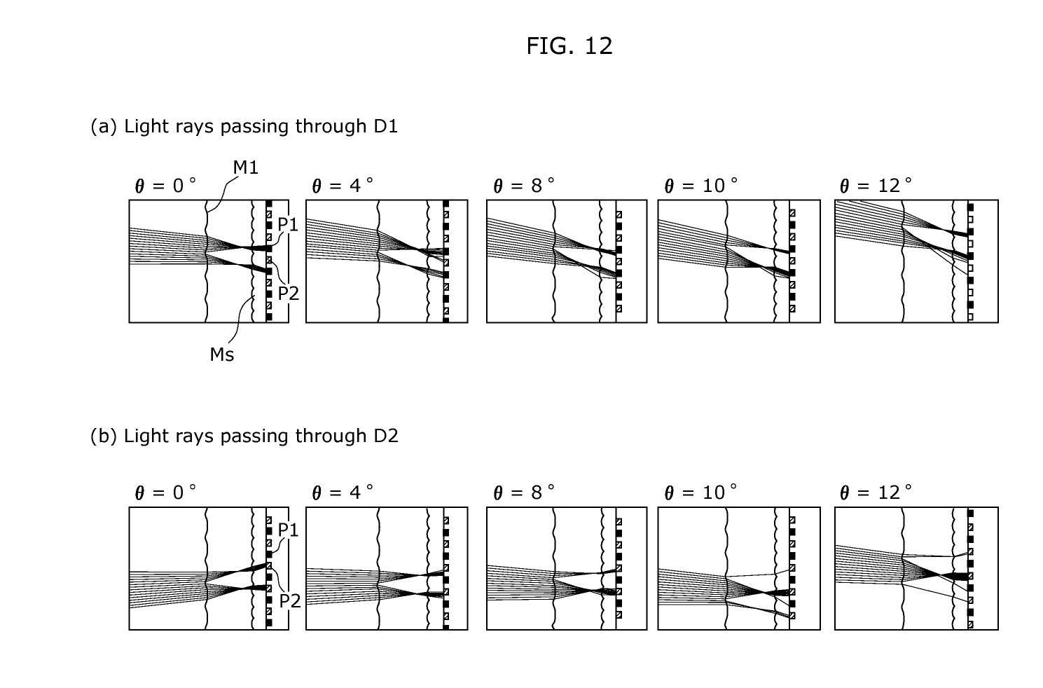

FIG. 12 is a diagram showing analyses of paths of light fluxes including the chief ray CR entering the lenticular lens M3 at an incident angle θ. FIG. 12 shows only the representative light rays including the chief ray CR.

The part (a) of FIG. 12 shows an analysis of the paths of light rays passing through the first region D1 of the first optical element L1. The part (b) of FIG. 12 shows an analysis of the paths of light rays passing through the second region D2 of the first optical element L1. The parts (a) and (b) of FIG. 12 show analyses when θ is 0°, 4°, 8°, 10°, or 12°.

Here, at the position at which the incident angle θ is 4°, 8°, 10°, or 12°, the offset value Δ of 9%, 20%, 25%, or 30% is correspondingly set for the pitch of the lenticular lens M3.

From FIG. 12, it is apparent that offsetting the optical components of the lenticular lens by the offset value Δ in relation to the pixel arrangement reduces the crosstalk when the incident angle θ is 8° or less.

As described above, with the imaging device A according to the present embodiment, providing the blazed diffraction grating M2 on the surface of the third optical element K on the object side enables, through the diffraction effect, reduction of the incident angle of the light rays entering the lenticular lens M3, thereby allowing the light rays to be closer to being parallel to the optical axis.

Furthermore, with the imaging device A according to the present embodiment, offsetting each optical component included in the lenticular lens M3 in relation to the arrangement of the corresponding first pixels P1 and second pixels P2 enables further reduction of the incident angle of the light rays entering the lenticular lens M3. As a result, the imaging device A according to the present embodiment can further reduce the crosstalk.

As an example, it is assumed that the refractive index n of the third optical element K is 1.526 and the depth of the diffraction grooves is 0.95 μm. With this, Equation 2 gives that m is approximately 1 for light having a wavelength of 500 nm. This means that the blazed diffraction grating M3 allows generation of the first-order diffracted light with a diffraction efficiency of approximately 100%.

Given that the pitch of the diffraction grating at a position at which the chief ray CR enters the blazed diffraction grating M3 is 7 μm, θb is about 8° when φ is 16°. That is to say, as compared to the comparable optical element shown in the part (a) of FIG. 8, the crosstalk can be reduced even when the incident angle φ of the chief ray CR entering the surface of the third optical element K on the object side increases by about 8°.

In other words, offsetting each optical component of the lenticular lens M3 in relation to the pixel arrangement as in the present embodiment enables reduction of the crosstalk even when the incident angle φ increases by degrees up to about 16°, thereby enabling a further increase in the design flexibility of the imaging device.

### Embodiment 3

Next, Embodiment 3 of the present invention will be described.

The imaging device according to Embodiment 3 differs from the imaging devices according to Embodiments 1 and 2 mainly in the following points: Firstly, the first point is that the first optical element L1 has four regions having different optical properties. Next, the second point is that a microlens array, rather than the lenticular lens, is formed on one of the surfaces of the third optical element K. Lastly, the third point is that the blazed diffraction grating has a concentric structure in relation to the optical axis. Referring to the drawings, the following describes Embodiment 3, centering on the points different from Embodiments 1 and 2.

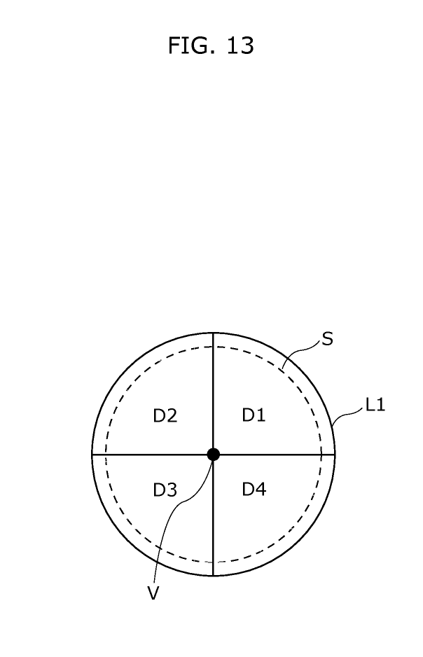

FIG. 13 is a front view of the first optical element L1 according to present embodiment as seen from the object side. A first region D1, a second region D2, a third region D3, and a fourth region D4 are four vertically and horizontally separated regions with the optical axis V as the center of the boundaries.

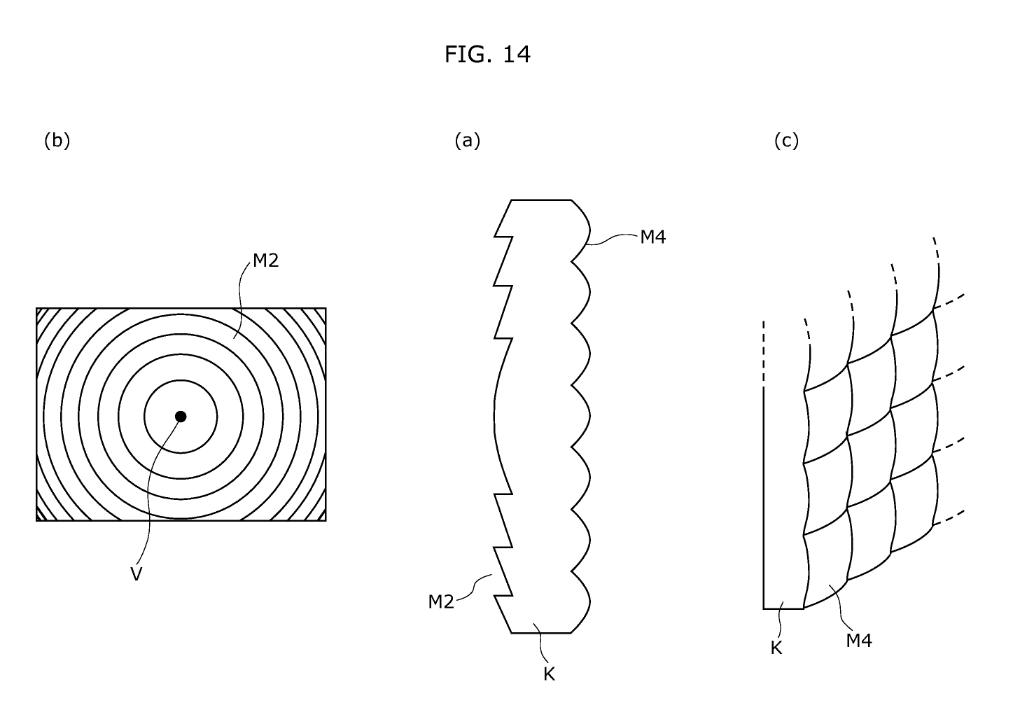

FIG. 14 is a configuration diagram of the third optical element K according to the present embodiment. More specifically, the part (a) of FIG. 14 is a cross-section diagram of the third optical element K. The part (b) of FIG. 14 is a front view of the third optical element K seen from the blazed diffraction grating M2 side. The part (c) of FIG. 14 is a partial enlarged perspective view of the third optical element K seen from the microlens array M4 side.

As shown in FIG. 14, a microlens array M4 having a plurality of microlenses is formed on a surface of the third optical element K on the imaging element N1 side. Furthermore, the blazed diffraction grating M2 including concentric diffractive ring zones having the optical axis V as their center is formed on a surface of the third optical element K on the lens optical system L side (i.e., on the object side). It is to be noted that the shape and exact size of the pitch of each of the microlens array M4 and the blazed diffraction grating M2 will not be described because it is sufficient as long as they are determined according to the function or use purpose of the imaging device A.

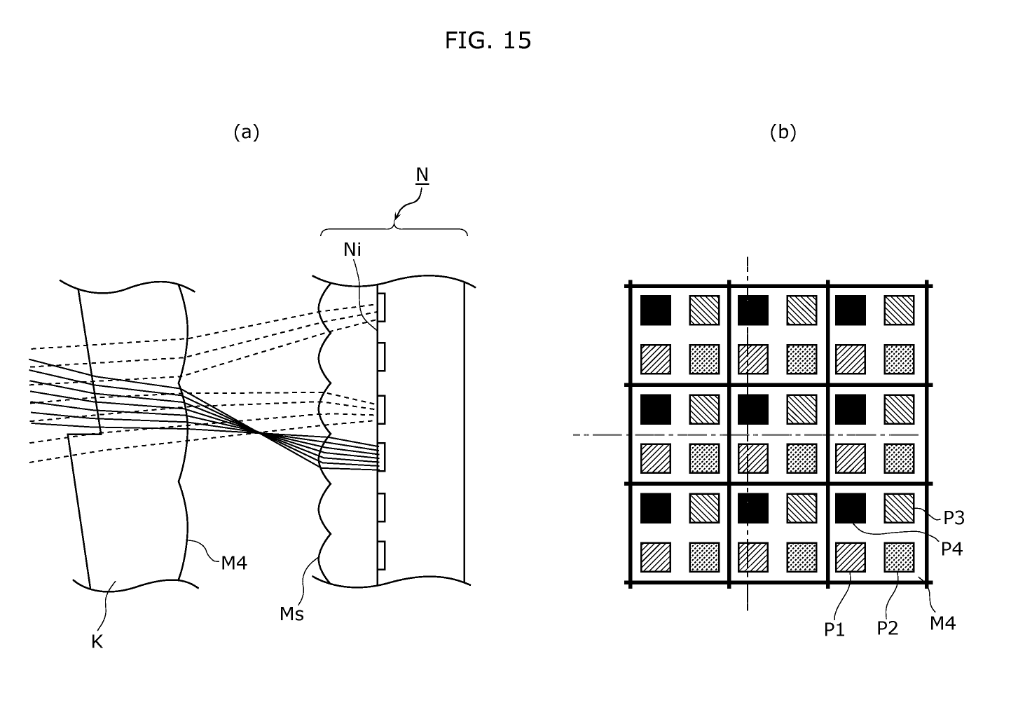

FIG. 15 is a diagram for describing the positional relationship between the third optical element K and pixels on the imaging element N. More specifically, the part (a) of FIG. 15 is an enlarged view of the third optical element K and the imaging element N. The part (b) of FIG. 15 is a diagram showing the positional relationship between the third optical element K and pixels on the imaging element N.

As in Embodiment 1, the third optical element K is provided near the focal point of the lens optical system L and is provided at a position away from the image plane Ni by a predetermined distance. Furthermore, pixels are arranged in rows and columns on the image plane Ni of the imaging element N. Each of these pixels arranged in such a manner can be classified as a first pixel P1, a second pixel P2, a third pixel P3, or a fourth pixel P4. Moreover, a microlens Ms is provided over the pixels.

Furthermore, the microlens array M4 is formed on the surface of the third optical element K on the imaging element N side. The microlens array M4 is equivalent to an arrayed optical element. Each of microlenses (optical components) included in the microlens array M4 corresponds to one of sets of four pixels, namely, the first pixel P1, the second pixel P2, the third pixel P3, and the fourth pixel P4 that are arranged in rows and columns on the image plane Ni.

Such a structure allows most part of the light fluxes passing through the first region D1, the second region D2, the third region D3, and the fourth region D4 on the first optical element L1 shown in FIG. 13 to reach the first pixel P1, the second pixel P2, the third pixel P3, and the fourth pixel P4 on the image plane Ni, respectively.

Here, the signal processing unit C generates the object information using first pixel values obtained from the first pixels P1, second pixel values obtained from the second pixels P2, third pixel values obtained from the third pixels P3, and fourth pixel values obtained from the fourth pixels P4. As in Embodiment 1, the signal processing unit C according to the present embodiment generates, as the object information, a first image I1 including the first pixel values, a second image I2 including the second pixel values, a third image I3 including the third pixel values, and a fourth image I4 including the fourth pixel values.

Next, a method for determining an object distance from the object information will be described as an example of use of the object information.

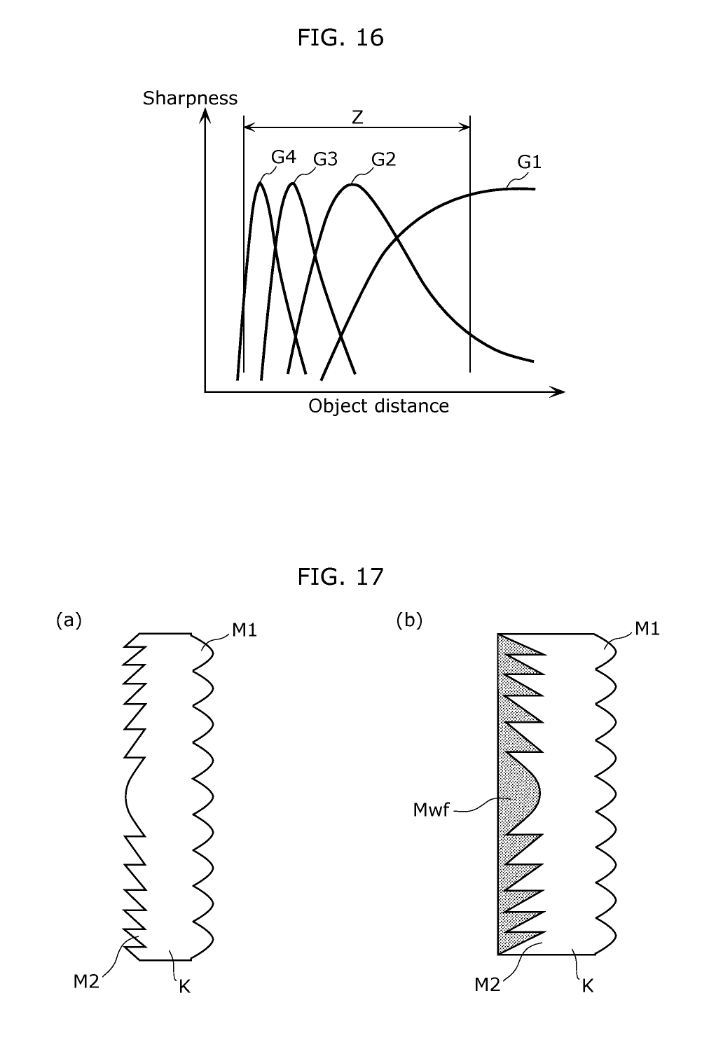

In this example, it is assumed that the first region D1, the second region D2, the third region D3, and the fourth region D4 have optical properties that make the focus properties of the passing light rays different from each other. More specifically, a flat lens is provided as the first region D1, a spherical lens having a curvature radius of R2 is provided as the second region D2, a spherical lens having a curvature radius of R3 is provided as the third region D3, and a spherical lens having a curvature radius of R4 is provided as the fourth region D4 (R2>R3>R4), for example. The optical axes of the spherical lenses of the second region D2, the third region D3, and the fourth region D4 match the optical axis V of the lens optical system L described earlier. FIG. 16 is a graph showing the relationship between the object distance and the sharpness in this case. In the graph shown in FIG. 16, a profile G1 shows the sharpness of a predetermined region of the image generated using only the pixel values of the first pixels P1. A profile G2 shows the sharpness of a predetermined region of the image generated using only the pixel values of the second pixels P2. A profile G3 shows the sharpness of a predetermined region of the image generated using only the pixel values of the third pixels P3. A profile G4 shows the sharpness of a predetermined region of the image generated using only the pixel values of the third pixels P4.

Furthermore, a range Z is a range in which the sharpness according to the profile G1, G2, G3, or G4 varies in response to a change in the object distance. Thus, the object distance can be determined using such a relationship in the range Z.

For example, in the range Z, at least one of a sharpness ratio between the profile G1 and the profile G2 and a sharpness ratio between the profile G3 and the profile G4 is correlated with the object distance. In view of this, use of such a correlation enables determination of the object distance for each of the predetermined regions of the respective images based on these sharpness ratios.

It is to be noted that the optical properties different between the first region D1, the second region D2, the third region D3, and the fourth region D4 are not limited to the above-described example. Depending on what kind of optical properties are to be made different, the use of the object information changes. The method for determining the object distance such as the above-described method is an example of use of the object information. For example, a sum image I5, which is a sum of the first image I1, the second image I2, the third image I3, and the fourth image I4, may be generated. The sum image I5 generated in this manner is an image larger in depth of field than the first image I1, the second image I2, the third image I3, and the fourth image I4.

Furthermore, the object distance can be determined for each of the predetermined regions of the respective images using a ratio between the sharpness of a predetermined region of the sum image I5 and the sharpness of a predetermined region of the first image I1, the second image I2, the third image I3, or the fourth image I4.

It is to be noted that the signal processing unit C may determine the object distance or generate the sum image I5 and so on, using the object information as described above.

As described thus far, the imaging device A according to the present embodiment can increase the design flexibility and reduce the crosstalk as well as being capable of generating four images in one imaging operation using a single imaging optical system.

### Embodiment 4

Next, Embodiment 4 of the present invention will be described.

Embodiment 4 is different from the other embodiments in that the blazed diffraction grating has two layers. The following description centers on the points different from Embodiments 1 to 3 and omits a detailed description of the points in common with Embodiments 1 to 3.

The part (a) of FIG. 17 is a cross-section diagram of the third optical element K according to Embodiment 1. According to Embodiment 1, the lenticular lens M1 having an arc cross section is formed on the surface of the third optical element K on the imaging element N1 side, and the blazed diffraction grating M2 is formed on the lens optical system L side (i.e., on the object side).

In contrast, the part (b) of FIG. 17 is a cross-section diagram of the third optical element K according to the present embodiment. According to the present embodiment, a cover film Mwf is provided on the blazed diffraction grating M2 formed on the surface of the third optical element K on the lens optical system L side. That is to say, the third optical element K includes the cover film Mwf covering the blazed diffraction grating M2.

When the d-line refractive index of the blazed diffraction grating M2 is n1 and the d-line refractive index of the cover film is n2, these refractive indices represent functions of a wavelength λ. More specifically, when the depth d′ of the diffraction grooves approximately satisfies the following Equation 3 in all visible wavelength bands, the m-th order (or the negative m-th order when the slope direction of the blaze is reversed between left and right) diffraction efficiency is independent of the wavelength and is approximately 100%. It is to be noted that m represents the diffraction order.

d′=mλ/|n1−n2| (Equation 3)

It is to be noted that the meaning of “approximately satisfies Equation 3” includes satisfying Equation 3 in a range which can be regarded as substantially identical to the range in which Equation 3 is strictly satisfied.

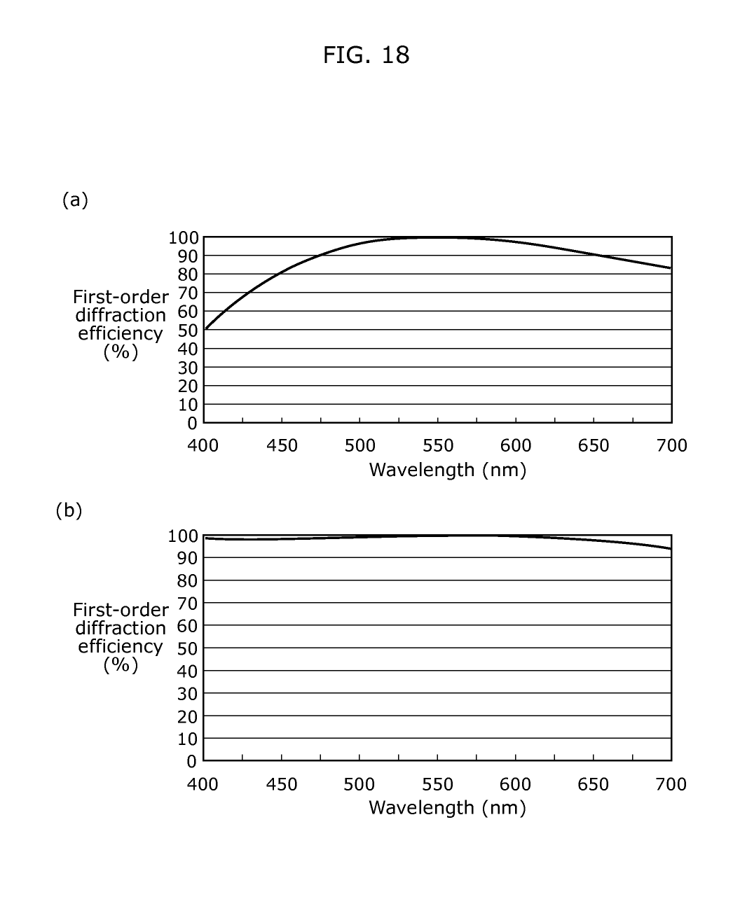

The part (a) of FIG. 18 is a graph showing the relationship between wavelength and the first-order diffraction efficiency of the blazed diffraction grating M2 according to Embodiment 1. More specifically, the part (a) of FIG. 18 shows wavelength dependency of the first-order diffraction efficiency with respect to light rays vertically entering the blazed diffraction grating M2.

As for the part (a) of FIG. 18, a base material having a d-line refractive index of 1.52 and an Abbe number of 56 is used as the base material of the blazed diffraction grating M2. Furthermore, the depth of the diffraction grooves of the blazed diffraction grating M2 is 1.06 μm.

In contrast, the part (b) of FIG. 18 is a graph showing the relationship between wavelength and the first-order diffraction efficiency of the blazed diffraction grating M2 according to the present embodiment. More specifically, the part (b) of FIG. 18 shows wavelength dependency of the first-order diffraction efficiency with respect to light rays vertically entering the blazed diffraction grating M2.

As for the part (b) of FIG. 18, a polycarbonate (d-line refractive index: 1.585, Abbe number: 28) is used as the base material of the blazed diffraction grating M2. Furthermore, used as the cover film Mwf is a resin (d-line refractive index: 1.623, Abbe number: 40) made by dispersing, in an acrylic ultraviolet curable resin, a zirconium oxide having a particle size of 10 nm or less. Given this, the right side of Equation 3 is approximately constant regardless of the wavelength. It is to be noted that the depth d′ of the diffraction grooves of the blazed diffraction grating M2 is 15 μm.

Providing the cover film Mwf to cover the blazed diffraction grating M2 formed on the third optical element K as in the present embodiment increases the first-order diffraction efficiency to approximately 100% in all visible wavelength bands as shown in the part (b) of FIG. 18. In addition, when d′=30 μm, the second-order diffraction efficiency can also be increased to approximately 100%.

As described thus far, with the imaging device according to the present embodiment, it is possible to achieve a high diffraction efficiency in all visible wavelength bands by covering the blazed diffraction grating M2 with the cover film Mwf in such a manner that Equation 3 is approximately satisfied.

It is to be noted that the combination of materials of the third optical element K and the cover film is not limited to the materials described above, and materials such as various types of glass, various types of resin, or a nanocomposite material may be combined. This enables implementation of the imaging device capable of capturing a bright image with less light loss.

### Embodiment 5

Next, Embodiment 5 of the present invention will be described.

The imaging device according to Embodiment 5 is different from the imaging devices according to Embodiments 1 to 4 in that the third optical element K including the blazed diffraction grating and either the lenticular lens or the microlens array is integrally formed with the imaging element N. The following description centers on the points different from Embodiments 1 to 4 and omits a detailed description of the points in common with Embodiments 1 to 4.

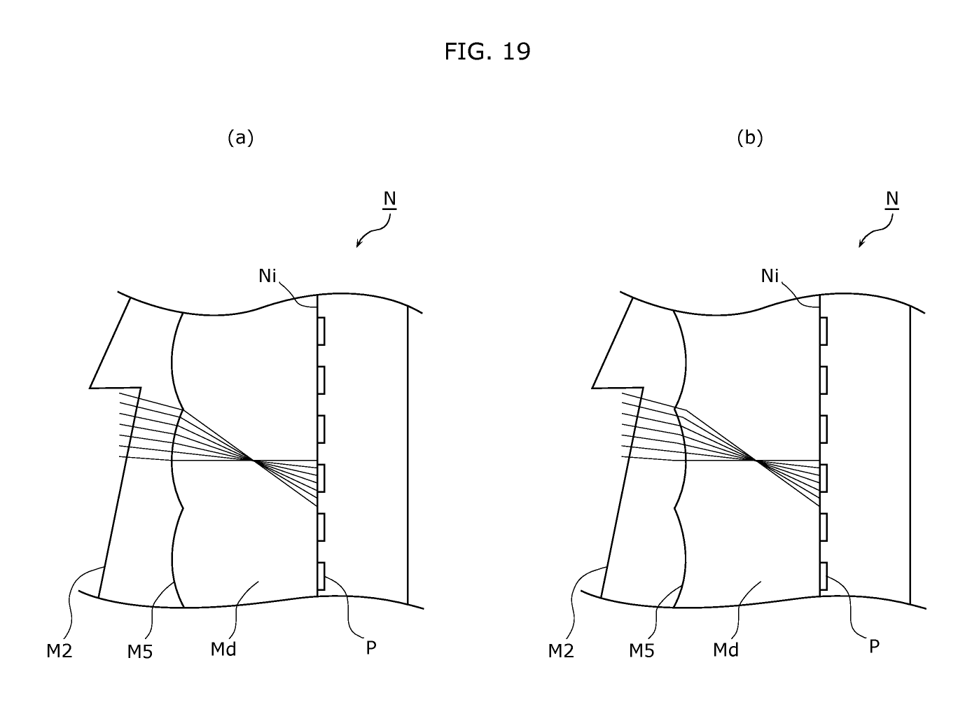

FIG. 19 is an enlarged cross-section diagram of the third optical element K and the imaging element N according to Embodiment 5. In the present embodiment, the third optical element K including the blazed diffraction grating M2 and a lenticular lens (or microlens array) M5 is integrated with the imaging element N via a medium Md. As in the embodiments such as Embodiment 1, pixels P are arranged in rows and columns on the image plane Ni. One of the optical components of the lenticular lens or one of the microlenses of the microlens array corresponds to a set of pixels among these pixels P.

In the part (a) of FIG. 19, the third optical element K and the imaging element N are integrated in such a manner that each optical component of the lenticular lens (or microlens array) M5 has a convex surface on the object side. In such a case, the medium Md between the third optical element K and the imaging element N includes a material higher in refractive index than the third optical element K (a medium between the blazed diffraction grating M2 and the lenticular lens (or microlens array) M5). For example, it is sufficient as long as the third optical element K includes SiO2 and the medium Md includes SiN.

It is to be noted that the third optical element K and the imaging element N may be integrated in such a manner that each optical component of the lenticular lens (or microlens array) M5 has a concave surface on the object side as shown in the part (b) of FIG. 19. In such a case, the medium Md between the third optical element K and the imaging element N includes a material lower in refractive index than the third optical element K (the medium between the blazed diffraction grating M2 and the lenticular lens (or microlens array) Md).

Even in the present embodiment, the light fluxes passing through different regions of the first optical element L1 can be guided to different pixels as in Embodiments 1 to 4.

Next, the following describes, as a variation of the present embodiment, a case where a microlens Ms is provided on the image plane Ni.

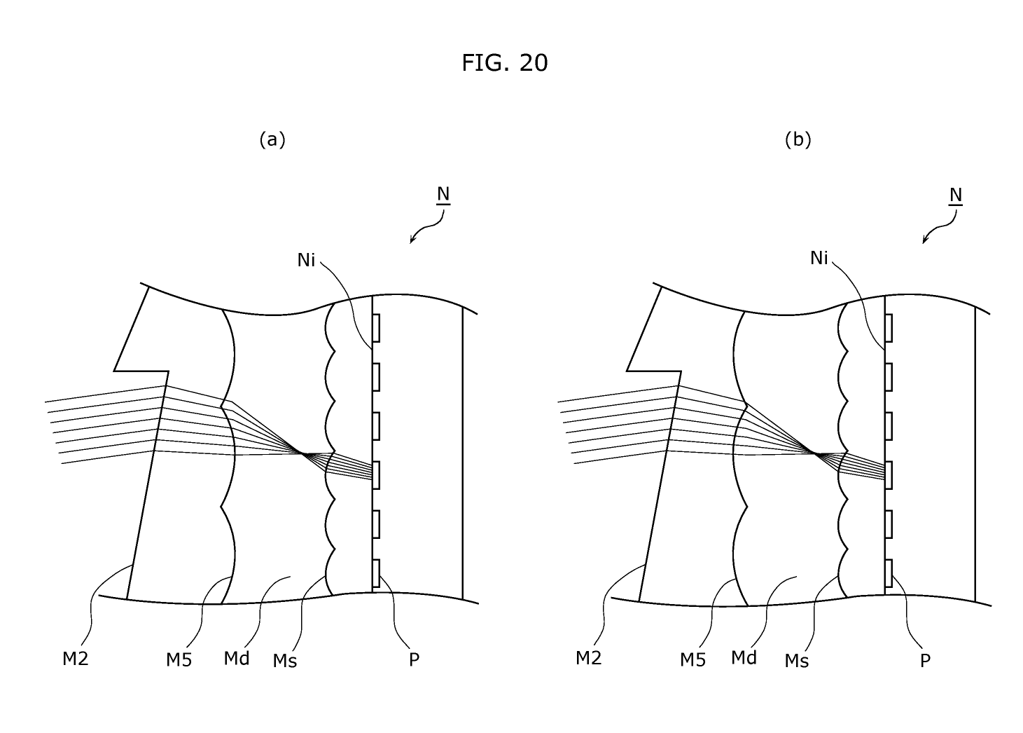

FIG. 20 is an enlarged cross-section diagram of the third optical element K and the imaging element N according to a variation of Embodiment 5. In the present variation, the microlens Ms is formed on the image plane Ni to cover the pixels P, and the medium Md and the third optical element K are stacked over the microlens Ms.

In the part (a) of FIG. 20, the third optical element K and the imaging element N are integrated in such a manner that each optical component of the lenticular lens (or microlens array) M5 has a concave surface on the object side. In such a case, the medium Md between the lenticular lens (or microlens array) M5 and the microlens Ms includes a material lower in refractive index than either the third optical element K (the medium between the blazed diffraction grating M2 and the lenticular lens (or microlens array) M5) or the microlens Ms. For example, it is sufficient as long as the third optical element K and the medium Md include a resin material when the microlens Ms includes a resin material.

It is to be noted that the third optical element K and the imaging element N may be integrated in such a manner that each optical component of the lenticular lens (or microlens array) M5 has a convex surface on the object side as shown in the part (b) of FIG. 20. In such a case, the third optical element K (the medium between the blazed diffraction grating M2 and the lenticular lens (or microlens array) M5), the medium Md between the microlens Ms and the lenticular lens (or microlens array) M5, and the microlens Ms include materials having refractive indices that increase in the order mentioned. It is to be noted that by providing the microlens Ms over the pixels, the light collection efficiency can be increased in the present variation as compared to Embodiment 5.

It is to be noted that providing the cover film covering the third optical element K and the blazed diffraction grating using a combination of materials having refractive indices approximately satisfying Equation 3 as shown in Embodiment 4 enables implementation of the imaging device capable of capturing a bright image with less light loss in all visible wavelength bands.

As described thus far, the imaging device A according to the present embodiment or its variation allows integration of the third optical element K and the imaging element N. When the third optical element K and the imaging element are separate as in Embodiments 1 to 4, the alignment of the third optical element K and the imaging element N is difficult. On the other hand, integrally forming the third optical element K and the imaging element N as in the present embodiment or its variation makes it possible to align the third optical element K and the imaging element N in the wafer processing, thereby simplifying the alignment and increasing the alignment precision.

Thus far, the imaging device A according to an aspect of the present invention has been described based on the embodiments, but the present invention is not limited to these embodiments. The scope of one or more aspects of the present invention is intended to include what is conceivable to those skilled in the art without materially departing from the novel teachings and advantages of the present invention, such as various modifications made to the embodiments and embodiments achieved by combining the structural elements in different embodiments.

For example, although the lens optical system L according to Embodiments 1 to 5 above is an image-side non-telecentric optical system, it may be an image-side telecentric optical system. In such a case, the imaging device A can further reduce the crosstalk.

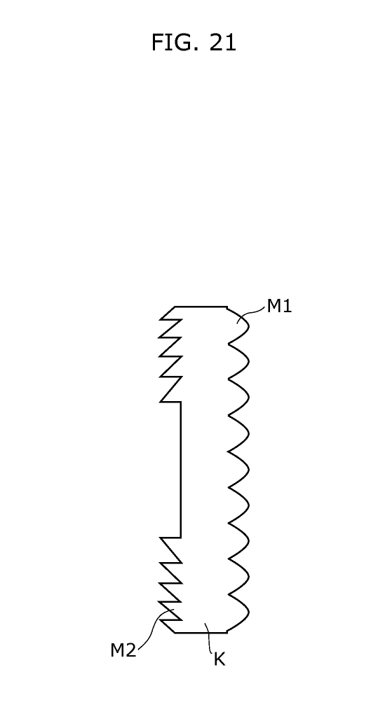

Furthermore, although the blazed diffraction grating M2 according to Embodiments 1 to 5 above is formed on the entire surface of the third optical element K on the object side, it does not necessarily have to be formed on the entire surface. The incident angle φ of the chief ray CR entering the surface of the third optical element K on the object side varies depending on the distance H from the optical axis V. In a typical lens optical system, the incident angle φ increases with the distance H. In view of this, it is sufficient as long as the blazed diffraction grating M2 is formed at least at a position away from the optical axis V (i.e., a position at which the incident angle φ increases). In other words, the blazed diffraction grating M2 does not necessarily have to be formed near the optical axis V. That is to say, the blazed diffraction grating M2 according to Embodiments 1 to 5 above may be formed only in regions away from the optical axis V by a predetermined distance or longer (peripheral areas) as shown in FIG. 21. This allows the central part of the third optical element K to be flat, simplifying the manufacture of the third optical element K.

Moreover, the blazed diffraction grating M2 may be formed to have a pitch P that decreases in the peripheral areas in which the angle φ increases. This enables reduction of θb in the peripheral areas of the blazed diffraction grating M2, in which the incident angle φ increases.

Furthermore, the blazed diffraction grating M2 may be formed to have a depth d of the diffraction grooves that increases in the peripheral areas. This allows an increase in the diffraction order m of the peripheral areas of the blazed diffraction grating M2, thereby allowing further reduction of θb.

Furthermore, the description in Embodiments 1 to 5 has centered on the case where the regions of the first optical element L1 have different focus properties. However, the regions of the first optical element L1 do not necessarily have to have different focus properties.

For example, the first optical element L1 may have regions having different light transmittance. More specifically, neutral density (ND) filters having different light transmittance may be provided in the regions. In this case, the imaging device A can generate, in one imaging operation, an image of a dark object from light rays passing through a region of a high transmittance and an image of a bright object from light rays passing through a region of a low transmittance. Furthermore, by combining these images, the imaging device A can generate an image having a wide dynamic range.

Alternatively, the first optical element L1 may have regions that transmit light rays at different wavelength bands. More specifically, filters having different transmitting wavelength bands may be provided in the regions. In this case, it is possible to generate, in one imaging operation, a color image at the visible light wavelengths and an image at the near-infrared wavelengths, for example. To give an example, a single imaging device can capture a color image at the daytime and a dark image at the nighttime without having to switch functions between the daytime and the nighttime.

Moreover, although the blazed diffraction grating is formed on the third optical element K according to Embodiments 1 to 5 above, a different diffraction grating symmetric about the optical axis V may be formed.

### INDUSTRIAL APPLICABILITY

The imaging device according to an aspect of the present invention is useful as a digital still camera or a digital video camera, for example. It can also be used as: an in-vehicle camera; a security camera; a camera for medical use such as for an endoscope or a capsule endoscope; a camera for biometric authentication; a camera for a microscope; or a camera for an astronomical telescope and the like used for obtaining a spectral image.

### REFERENCE SIGNS LIST

A Imaging device

L Lens optical system

L1 First optical element

L2 Second optical element

D1 First region

D2 Second region

D3 Third region

D4 Fourth region

S Diaphragm

K Third optical element

N Imaging element

Ni Image plane

Ms Microlens

M1, M3, M5 Lenticular lens

M2 Blazed diffraction grating

M4 Microlens array

Mwf Cover film

CR Chief ray

H Distance

P Pixel

P1 First pixel

P2 Second pixel

P3 Third pixel

P4 Fourth pixel

C Signal processing unit

1. An imaging device comprising:
a lens optical system including at least a first region and a second region having different optical properties;
an imaging element including at least first pixels and second pixels which light passing through the lens optical system enters;
an arrayed optical element which is provided between the lens optical system and the imaging element, allows light passing through the first region to enter the first pixels, and allows light passing through the second region to enter the second pixels;
a signal processing unit configured to generate object information using first pixel values obtained from the first pixels and second pixel values obtained from the second pixels; and
a diffractive optical element provided between the arrayed optical element and the lens optical system and including a diffraction grating symmetrical about an optical axis of the lens optical system.

2. The imaging device according to claim 1,
wherein the lens optical system includes (i) a diaphragm having an opening in a region including the optical axis and (ii) an optical element provided near the diaphragm and including at least the first region and the second region.

3. The imaging device according to claim 1,
wherein the diffraction grating is formed only in a region away from the optical axis by a predetermined distance or longer.

4. The imaging device according to claim 1,
wherein each of the first pixels is adjacent to at least one of the second pixels.

5. The imaging device according to claim 1,
wherein the first pixels and the second pixels are alternately arranged.

6. The imaging device according to claim 1,
wherein the arrayed optical element includes optical components each being offset in relation to arrangement of a corresponding at least one of the first pixels and a corresponding at least one of the second pixels.

7. The imaging device according to claim 1,
wherein the lens optical system is an image-side non-telecentric optical system.

8. The imaging device according to 7 claim 1,
wherein the first pixels are arranged in a row in a horizontal direction, and the second pixels are arranged in a row in the horizontal direction, and
the first pixels and the second pixels are alternately arranged in a vertical direction.

9. The imaging device according to claim 8,
wherein the arrayed optical element is a lenticular lens,
the lenticular lens includes horizontally elongate optical components arranged in the vertical direction, and
each of the optical components corresponds to two rows of pixels, which are one row of the first pixels and one row of the second pixels.

10. The imaging device according to claim 1,
wherein the lens optical system further includes a third region and a fourth region,
the first, second, third, and fourth regions have different optical properties,
the imaging element further includes third pixels and fourth pixels which the light passing through the lens optical system enters,
the arrayed optical element further allows light passing through the third region to enter the third pixels, and allows light passing through the fourth region to enter the fourth pixels, and
the signal processing unit is configured to generate the object information using the first pixel values, the second pixel values, third pixel values obtained from the third pixels, and fourth pixel values obtained from the fourth pixels.

11. The imaging device according to claim 10,
wherein one of the first pixels, one of the second pixels, one of the third pixels, and one of the fourth pixels make up one of sets of four pixels and are arranged in two rows and two columns.

12. The imaging device according to claim 11,
wherein the arrayed optical element is a microlens array,
the microlens array includes optical components, and
each of the optical components corresponds to one of the sets of four pixels.

13. The imaging device according to claim 1,
wherein the diffraction grating is a blazed diffraction grating.

14. The imaging device according to claim 13,
wherein the diffractive optical element includes a cover film covering the blazed diffraction grating, and
the blazed diffraction grating includes diffraction grooves having a depth d′ which satisfies d′=mλ/|n1−n2| in all visible wavelength bands when n1 is a d-line refractive index of the blazed diffraction grating, n2 is a d-line refractive index of the cover film, and m is a positive integer.

15. The imaging device according to claim 1,
wherein the diffractive optical element and the arrayed optical element are integrally formed.

16. The imaging device according to claim 1,
wherein the arrayed optical element is integrally formed with the imaging element.

17. The imaging device according to claim 16, further comprising
a microlens provided between the arrayed optical element and the imaging element,
wherein the arrayed optical element is integrally formed with the imaging element via the microlens.
